(19) United States
(12) Patent Application Publication
(10) Pub. No.: US 2015/0328202 A1
(43) Pub. Date: Nov. 19, 2015

(54) EDIBLE PRODUCT FOR NICOTINE DELIVERY

(71) Applicant: Idea Logic LLC, Somerset, NJ (US)

(72) Inventor: Andreas Balogh, Somerset, NJ (US)

(21) Appl. No.: 14/714,276

(22) Filed: May 16, 2015

Related U.S. Application Data
(60) Provisional application No. 61/994,266, filed on May 16, 2014.

(51) Int. Cl. A61K 31/465

(52) U.S. Cl. CPC A61K 31/465

(57) ABSTRACT

An edible product for oral ingestion comprising one or more edible seeds infused with nicotine is disclosed. The edible product enables reduction in nicotine craving in nicotine addicted persons. A therapeutic package for use with one or more edible seeds is also disclosed, comprising a first predefined quantity of powdered nicotine; and a second predefined quantity of at least one control agent, wherein the powdered nicotine and the control agent are infused in the edible seeds for oral ingestion A method for administration of nicotine as a medical therapy to a person is also disclosed, comprising providing a dose of one or more edible seeds infused with nicotine to the person; and periodically repeating the dose over predefined time intervals to achieve a predefined level of nicotine in bloodstream of the person.

### CROSS-REFERENCE TO RELATED APPLICATIONS

This application claims the benefit of U.S. Provisional Application Ser. No. 61/994,266 filed May 16, 2014, entitled “NICOTINE TREATED EDIBLE SEEDS,” which is incorporated herein by reference in its entirety.

### BACKGROUND

1. Field

Embodiments of the present invention generally relate to delivery of an effective amount of nicotine to a person and in particular a product, package, and method of administering nicotine through oral ingestion for therapeutic purposes.

2. Description of Related Art

Generally cigars, cigarettes, and other smoking items include tobacco that is a source of nicotine. These smoking items are a source of inhalation-related diseases, which are generally resulted from non-nicotine based constituents in the smoked material. While, nicotine is a stimulant or a psychoactive compound that can enhance the activity of central and peripheral nervous system, it is also addictive if not taken carefully in controlled doses. As a result, smokers find abstinence difficult and may suffer from withdrawal symptoms, for example, anxiety.

With the increasing recognition of the health hazards associated with smoking, for example, smoking cigarettes, increasing attention has been focused on less harmful or alternative forms of nicotine administration to provide the effectiveness and satisfaction of nicotine. For example, smokeless tobacco products have been developed that can be chewed or ingested to absorb nicotine. Therefore, by temporarily giving a smoker an alternative source of absorbing nicotine, medical conditions can be helped, withdrawal symptoms can be relieved, and smoking abstinence facilitated.

In addition to smoking cessation, alternative forms of nicotine administration may have applicability in long-term maintenance, to reduce, if not entirely eliminate, the harmful effects resulting from smoke inhalation-related diseases. However, smokeless tobacco products generally suffer from first-pass effect of human metabolism that reduced the effectiveness of such products after passing through the liver of a user.

Thus, there is a need for conveniently dispensed and effectively absorbable nicotine that can be administered to a person.

### SUMMARY

Embodiments in accordance with the present invention provide an edible product for oral ingestion comprising one or more edible seeds infused with nicotine.

Embodiments in accordance with the present invention further provide a therapeutic package for use with one or more edible seeds. The package comprises a first predefined quantity of powdered nicotine. Further, the package comprises a second predefined quantity of at least one control agent, wherein the powdered nicotine and the control agent are infused in the edible seeds for oral ingestion.

Embodiments in accordance with the present invention further provide a method for administration of nicotine as a medical therapy to a person. The method comprises providing a dose of one or more edible seeds infused with nicotine to the person. The method further comprises periodically repeating the dose over predefined time intervals to achieve a predefined level of nicotine in bloodstream of the person.

Embodiments in accordance with the present invention further provide a method for forming an edible product for oral ingestion. The method comprises mixing a predefined quantity of a therapeutic package with a predefined quantity of edible seeds. The method further comprises allowing the therapeutic package to be infused in the edible seeds for a predefined time duration.

Embodiments of the present invention may provide a number of advantages. First, the present invention enables reduction in nicotine craving in nicotine addicted individuals. Next, the present invention assists in reducing anxiety in humans. For example, the anxiety may be associated with nicotine withdrawal. Further, the present invention enables sufficient administration of nicotine to the human's metabolism after it passes through the portal vein entry of the liver, so the nicotine achieves a desired blood level in the human.

These and other advantages will be apparent from the present application of the embodiments described herein.

The preceding is a simplified summary to provide an understanding of some embodiments of the present invention. This summary is neither an extensive nor exhaustive overview of the present invention and its various embodiments. The summary presents selected concepts of the embodiments of the present invention in a simplified form as an introduction to the more detailed description presented below. As will be appreciated, other embodiments of the present invention are possible utilizing, alone or in combination, one or more of the features set forth above or described in detail below.

The headings used herein are for organizational purposes only and are not meant to be used to limit the scope of the description or the claims. As used throughout this application, the word _{may} is used in a permissive sense (i.e., meaning having the potential to), rather than the mandatory sense (i.e., meaning must). Similarly, the words “include”, “including”, and “includes” mean including but not limited to. To facilitate understanding, like reference numerals have been used, where possible, to designate like elements common to the figures. Optional portions of the figures may be illustrated using dashed or dotted lines, unless the context of usage indicates otherwise.

### DETAILED DESCRIPTION

In the following detailed description, numerous specific details are set forth in order to provide a thorough understanding of embodiments or other examples described herein. In some instances, well-known methods, procedures and compositions have not been described in detail, so as to not obscure the following description.

Further, the examples disclosed are for exemplary purposes only and other examples may be employed in lieu of, or in combination with, the examples disclosed. It should also be noted the examples presented herein should not be construed as limiting of the scope of embodiments of the present disclosure, as other equally effective examples are possible and likely.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B, and C together.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

In an embodiment of the present invention, an edible product may be used for nicotine administration. The edible product may include one or more edible seeds infused with nicotine. In an embodiment, the edible seeds belong to plant genus or family such as helianthus, cucurbita, fabaceae, or a combination thereof. For example, the edible seeds may be sunflower seeds, pumpkin seeds, lentils, nuts, or any other edible plant or flower seeds. The nicotine may be infused in the endosperm that is a tissue of the edible seeds, in an embodiment. Therefore, the nicotine can be effectively administered.

The edible product may be ingested orally through mouth of a person, in an embodiment. In another embodiment, the edible product may be administration to the esophagus, stomach, and/or duodenum, such as by way of ingesting whole, partially chewed, or chewed edible seeds. For example, the person can chew, suck, or swallow the nicotine infused edible seeds.

In an embodiment, the edible seeds are infused with one or more flavorings. In an embodiment, the flavorings may be fruit flavors, dairy product flavors, alcohol flavors, artificial edible flavors, other natural edible flavors, or a combination thereof. For example, flavorings include, but are not limited to, sugar, coffee, beer, wine, vodka, whiskey, fruit juice, milk, soda, and the like. Flavors of fruit juice include, for example, but are not limited to cranberry juice, grapefruit juice, lime juice, and orange juice and the like. Further, flavors of soda include, for example, but are not limited to root beer, cola, ginger ale, Sprite®, Dr. Pepper®, and the like. The flavorings make the edible product and the seeds more palatable. In an embodiment, the quantity of flavoring in the edible product is predefined as a ratio of the quantity of the edible product. Therefore, the quantity of flavoring increases or decreases with the quantity of the edible product. In another embodiment, the quantity and type of flavoring can be adjusted according to a personal preference of the person consuming the edible product. In an embodiment, multiple flavorings can be combined in the edible product.

The edible product may be provided in controlled doses to the person for effective absorption of nicotine in the bloodstream. In an embodiment, a predefined first quantity of nicotine per dose is at least about 0.5 milligram (mg), preferably in the range of about 1 mg to 42 mg, more preferable in the range of about 1 mg to 36 mg, and even more preferably in the range of about 2 mg to 26 mg, and most preferably in the range of about 4 mg to 12 mg.

In an embodiment, the quantity of the edible seeds ranges from about 5 grams to 20 grams per dose. In another embodiment, the quantity of edible seeds can be approximated based on a mouthful of seeds of the person ingesting the edible product. A person skilled in the art will appreciate that multiple or partial doses of the edible product can be administered as per requirement of nicotine level in the bloodstream of the person.

As discussed previously, the effectiveness of a dose may be affected by the first-pass effect, as the nicotine may be metabolized by the liver of the person. In an embodiment, the quantity of nicotine per dose is sufficient to obviate the problem of the first-pass absorption to the liver, so a therapeutically effective amount of the nicotine may be administered to the person's metabolism after pass through of the portal vein entry of the liver. Therefore, the quantity of nicotine provided by the edible product achieves a sufficient nicotine level in blood of the person in order to reduce the need of a smoker and assist in abstinence. Further, the administered nicotine level may be used to treat and alleviate anxiety, or other symptoms such as withdrawal symptoms in the person.

In an embodiment, a dosage frequency of the edible seeds may be about one dose per day, and preferably one dose in every predefined time intervals that ranges from about every one to two hours per day. For example, if one dose is administered at 9AM then the next dose may be administered at 10AM or 11AM. In an embodiment, the dose is provided only during the waking hours of the person. For instance, the waking hours may range from 7AM to 11 PM. A person skilled in the art will appreciate that the waking hours depend on biological clocks of the person, so may vary with every individual. In an embodiment, the dosing and the quantity of nicotine in the edible product may be adjusted for the individual person so the blood level of the nicotine does not rise above about a harmful amount. Further, the dosing may vary depending on the extent of a particular person's addiction to the smoking or nicotine-containing products.

In an embodiment, the dose achieves a therapeutic effect within a time range of about 30 minutes to 80 minutes from the ingestion of the edible product. In a more specific embodiment, the therapeutic effect is achieved within about 50 minutes from the ingestion of the edible product. The therapeutic effect may be associated with the predefined level of nicotine in the blood plasma or bloodstream of the person ingesting the edible product. In an embodiment, the predefined level of nicotine may be associated with a medicinally tolerable level of nicotine in a person. As discussed previously, the appropriate level of nicotine in the bloodstream may be specific to the individuals, and therefore, the exact dosage may vary from person to person.

In an embodiment of the present invention, a therapeutic package may be provided for use with edible seeds. The therapeutic package includes a first predefined quantity of powdered nicotine and a second predefined quantity of at least one control agent. The powdered nicotine and the control agent in the therapeutic package can be mixed with the edible seeds to infuse them with the nicotine. In an embodiment, the control agent may be used to control the chemical properties and/or proper infusion of the powdered nicotine in the edible seeds. A person skilled in the art will appreciate that various control agents that can be used for edible products are known. The present invention is not limited to use of any particular control agent.

In an embodiment, the therapeutic package may be in the form of a pouch or a container including powdered nicotine and the control agent as contents. Therefore, a person can pour the contents of the therapeutic package on a predefined quantity of edible seeds for infusing them with the contents. The edible seeds belong to plant genus or family such as helianthus, cucurbita, fabaceae, or a combination thereof. As also discussed previously, the powdered nicotine is infused in endosperm that is a tissue of the edible seeds. In an embodiment, the first predefined quantity of powdered nicotine may correspond to one dose of nicotine, as discussed previously. For example, the first predefined quantity may range about 4 mg to 12 mg per dose. In an embodiment, multiple therapeutic packages may be used to prepare one dose or multiple doses of the nicotine.

Further, the therapeutic package may include one or more flavorings as discussed previously. In an embodiment, the quantity and type of flavorings may be adjusted according to a personal preference of the person consuming the contents of the therapeutic package. In an embodiment, the dose contained in the therapeutic package achieves a therapeutic effect within a time range of about 30 minutes to 80 minutes, and more specifically within around 50 minutes from the ingestion of the infused edible seeds.

As also noted previously, a dosage frequency of the therapeutic package may be about one dose per day, and preferably one dose in the ranges of about every one to two hours per day to achieve the predefined nicotine level in the blood plasma of the person. As discussed above, the predefined level of nicotine may be associated with a medicinally tolerable level of nicotine in a person. In an embodiment, the dosage and the frequency may be adjusted according to the personal preference or medicinal effect on the person. The medicinal effect may be monitored by checking the nicotine level through a physician or by using test kits, in an embodiment.

In an embodiment of the present invention, the edible product for oral ingestion may be formed by mixing a predefined quantity of the contents of the therapeutic package with a predefined quantity of the edible seeds. Further, the contents of the therapeutic package may be allowed to be infused in the edible seeds for predefined time duration before administration, in an embodiment. For example, the contents of the therapeutic package may be poured on a predefined quantity of the edible seeds, mixed and left for a few minutes before ingestion the edible seeds. As a result, better effectiveness of the nicotine may be achieved.

Nicotine administration process as a medical therapy includes providing a dose of the edible seeds infused with nicotine to a person. Thereafter, the dose may be periodically repeated over predefined time intervals to achieve a predefined level of nicotine in bloodstream of the person, in an embodiment. As also noted previously, a dosage frequency may be about one dose per day, and preferably one dose in the ranges of about every one to two hours per day to achieve the predefined nicotine level in the blood plasma of the person. For example, the dose may be repeated every hour to provide therapeutic effect, such reducing the anxiety of the person. Further, the dose of administration includes nicotine in the range of 4 mg to 12 mg, in an embodiment.

In an embodiment, the administration may be orally through mouth of a person, through esophagus, stomach, and/or duodenum, such as by way of ingesting whole, partially chewed, or chewed edible seeds infused with nicotine. The edible seeds may be, for example but not limited to, sunflower seeds, pumpkin seeds, or any other edible seeds from plants or flowers.

The administration of the edible seeds as described in embodiments of the present invention enables sufficient and controlled delivery of nicotine in the bloodstream of the person. Further, the dosage can be adjusted based on the personal preference and medicine response of the person. Therefore, embodiments of the present invention can substantially alleviate the withdrawal symptoms associated with smoking abstinence, and further reduces the need of a smoker and assist in abstinence.

While the foregoing is directed to embodiments of the present invention, other and further embodiments of the present invention may be devised without departing from the basic scope thereof. It is understood that various embodiments described herein may be utilized in combination with any other embodiment described without departing from the scope contained herein. Further, the foregoing description is not intended to be exhaustive or to limit the invention to the precise form disclosed.

Modifications and variations are possible in light of the above teachings or may be acquired from practice of the invention. Certain exemplary embodiments may be identified by use of an open-ended list that includes wording to indicate the list items are representative of the embodiments and that the list is not intended to represent a closed list exclusive of further embodiments. Such wording may include “e.g.”, “etc.”, “such as”, “for example”, “and so forth”, “and the like”, etc., and other wording as will be apparent from the surrounding context.

No element, act, or instruction used in the description of the present application should be construed as critical or essential to the invention unless explicitly described as such. Also, as used herein, the article “a” is intended to include one or more items. Where only one item is intended, the term “one” or similar language is used. Further, the terms “any of” followed by a listing of a plurality of items and/or a plurality of categories of items, as used herein, are intended to include “any of,” “any combination of,” “any multiple of,” and/or “any combination of multiples of” the items and/or the categories of items, individually or in conjunction with other items and/or other categories of items.

Embodiments of the inventions set forth herein can be modified by one skilled in the art to achieve the benefit of the teachings as presented in the above descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Moreover, the claims should not be read as limited to the described order or elements unless stated to that effect. In addition, use of the term “means” in any claim is intended to invoke 35 U.S.C. §112, ¶6, and any claim without the word “means” is not so intended.

What is claimed is:

1. An edible product for oral ingestion comprising one or more edible seeds infused with nicotine.

2. The edible product of claim 1, wherein the one or more edible seeds belong to at least one of a plant genus helianthus, cucurbita, fabaceae, or a combination thereof.

3. The edible product of claim 1, wherein the nicotine is infused at least in endosperm of the edible seeds.

4. The edible product of claim 1, wherein the edible seeds are further infused with one or more flavorings.

5. The edible product of claim 4, wherein the flavorings are selected from a group consisting of fruit flavors, dairy product flavors, alcohol flavors, artificial edible flavors, and a combination thereof.

6. The edible product of claim 1, wherein the quantity of the edible seeds ranges from 10 grams to 20 grams per dose.

7. The edible product of claim 1, wherein the quantity of nicotine per dose ranges from 4 mg to 12 mg.

8. The edible product of claim 7, wherein the dose achieves a therapeutic effect within a time range of 30 minutes to 80 minutes from the ingestion.

9. The edible product of claim 1, wherein a dosage frequency of the edible seeds is one dose in the range of every one to two hours.

10. A therapeutic package for use with one or more edible seeds, the package comprising:
a first predefined quantity of powdered nicotine; and
a second predefined quantity of at least one control agent,
wherein the powdered nicotine and the control agent are infused in the edible seeds for oral ingestion.

11. The therapeutic package of claim 10, wherein the one or more edible seeds belong to at least one of a plant genus helianthus, cucurbita, fabaceae, or a combination thereof.

12. The therapeutic package of claim 10, wherein the nicotine is infused at least in endosperm of the edible seeds.

13. The therapeutic package of claim 10, further comprising one or more flavorings selected from a group consisting of fruit flavors, dairy product flavors, alcohol flavors, artificial edible flavors, and a combination thereof.

14. The therapeutic package of claim 10, wherein the first predefined quantity of nicotine ranges from 4 mg to 12 mg.

15. The therapeutic package of claim 10, wherein a therapeutic effect is achieved within a time range of 30 minutes to 80 minutes from the ingestion.

16. The therapeutic package of claim 10, wherein a dosage frequency is one dose in the range of every one to two hours.

17. A method for administration of nicotine as a medical therapy to a person, comprising:
providing a dose of one or more edible seeds infused with nicotine to the person; and
periodically repeating the dose over predefined time intervals to achieve a predefined level of nicotine in bloodstream of the person.

18. The method of claim 17, wherein the administration is performed orally through mouth of the person.

19. The method of claim 17, wherein the one or more edible seeds belong to at least one of a plant genus helianthus, cucurbita, fabaceae, or a combination thereof.

20. The method of claim 17, wherein the dose includes nicotine in the range of 4 mg to 12 mg.
